(19) United States
(12) Patent Application Publication
(10) Pub. No.: US 2010/0234339 A1
(43) Pub. Date: Sep. 16, 2010

(54) SILVER(I) COMPOUNDS AND THEIR USE IN PHARMACEUTICAL COMPOSITIONS FOR THE TREATMENT,PROPHYLAXIS AND PREVENTION OF INFECTIONS

(75) Inventors: Lars Öhrström, Gothenburg (SE); Vratislav Langer, Lerum (SE); Morsy A.M. Abu-Youssef, Alexandria (EG); Gohar Yoursy, Alexandria (EG); Alshima'a A. Massoud, Alexandria (EG)

Correspondence Address: HAMRE, SCHUMANN, MUELLER & LARSON, P.C., P.O. BOX 2902, MINNEAPOLIS, MN, 55402-0902, US

(73) Assignee: CSE INCUBATION AB

(21) Appl. No.: 12/451,739

(22) PCT Filed: Jun. 2, 2008
(86) PCT No.: PCT/EP2008/056773

§ 371 Date: May 24, 2010

(30) Foreign Application Priority Data
May 31, 2007 (SE) 0701314-7

(51) Int. Cl. A61K 31/555; C07F 15/00; A61P 31/00

(52) U.S. Cl. 514/188; 546/2

(57) ABSTRACT

The present invention provides new Ag(I) complexes with derivatives of nicotinic acid, nicotinamide and related pyridine ligands, and pharmaceutical compositions comprising such Ag(I) complexes for use in the treatment, prophylaxis and prevention of infections. The invention further provides a solvent free method for the synthesis of Ag(I) complexes containing substituted pyridines, pyrazines and pyrimidines as ligands.

### FIELD OF THE INVENTION

The present invention relates to new Ag(I) complexes with derivatives of nicotinic acid, nicotinamide and related pyridine ligands, and their use in antimicrobial compositions.

### BACKGROUND

The growing problem of antibiotic resistant bacterial strains makes it necessary for us to find new ways to fight bacteria and to prevent infections. In wound care medicine this is especially relevant for large burns, where bacterial infections may be lethal, and for chronic infections, for example those infections affecting diabetes patients.

Silver is recognised as a therapeutic agent used in the prevention of infection, and dilute solutions of silver nitrate and silver sulphadiazine have been among the most common preventive treatment for burns. The interest in silver is largely attributed to its bactericidal efficacy at low concentration and its relatively limited toxicity to human cells. Recently progress has led to an interest in dressings containing silver, which arises from advances in impregnation techniques and polymer technologies coupled with the increase in prevalence of bacterial resistance to antibiotics. There are now a number of silver-based dressings on the market that aim to improve healing primarily by controlling the wound bioburden.

Both the search for new more efficient antibacterial agents, and the concern for silver resistant bacteria, (Silver 2003, S. “Bacterial silver resistance: molecular biology and uses and misuses of silver compounds.” Ferns Microbiology Reviews 27(2-3): 341-353., Silver, S., L. T. Phung et al. 2006 “Silver as biocide in burns and wound dressing and bacterial resistance to silver compounds” Ind. Microbiol. Biotechnol. 33: 627-634.) make the development of new silver compounds as antibacterial agents an important challenge. Attention then needs to be directed to some of the possible limitations of the use of silver compounds, especially the precipitation of AgCl(s) when the agent comes into contact with the body fluids, and the photoinstability of many Ag (I) compounds. The reaction with serum albumin might also diminish the effectiveness of the Ag(I) compound.

Kitamura, K. and Kondo, Y. (JP 2000016906) describe a number of Ag(I) carboxylate complexes of nicotinic acid and related anionic ligands and their antibacterial properties. Chen, C-H, Cai, J. et al. 2002 (“Assembly via H-bonds and Ag—Ag attractions of one-dimensional silver(I) complexes of nicotinamide and nicotinic acid with sulfonate counter-anions” Polyhedron 21: 689-695) describe the crystal structure of certain nicontinamide and nicotinic acid Ag(I) complexes with sulfonates counter anions, no biological data of these complexes are presented.

Balakrishna, R. Bhogala, P. K. et al 2004 (“1:2 and 1:1 Ag(I)-isonicotinamide compounds: Five-fold interpenetrated CdSo4 network and the first example of (pyridine)N—Ag—O(amide) bonds” Crystal Growth & Design 4:215-218) describe the crystal structure of two Ag(I) isonicotinamide complexes, no biological data of these complexes are presented.

Dorn, T., Fromm, K M et al. 2006 (“[Ag(isonicotinamide)₂NO₃]₂ a stable form of silver nitrate” Aust. J. Chem 59: 22-25) describe physical properties of a Ag(I)-isonicotinamide complex, however, no data on antimicrobial effects are presented.

### BRIEF DESCRIPTION OF THE INVENTION

The present strategy to overcome the problems with solubility and photoinstability of Ag(I) compound is to investigate complexes having ligands that bind strongly to Ag(I), preventing premature release of “naked” Ag(T) and loss of efficiency due to insolubility and photoinstability. As the preferred coordination geometry of Ag (I) is linear two-coordinated and as it is known that ammonia dissolves precipitated AgCl(s) by forming a linear Ag(NH₃)₂⁺ complex, one should look at strongly binding nitrogen ligands enabling a linear coordination. Pyridine is the most well known such example, but of course the toxicity of this ligand makes it unsuitable.

However the related compounds nicotinic acid, nicotinamide (Vitamin B3) and their derivatives are known for their many beneficial uses. Nicotinic acid and nicotinamide are essential for the human body. Nicotinic acid lowers cholesterol and triglycerides, protects the body against atherosclerosis, and has antibacterial properties. (McPheat, W. L., Wardlaw, A. C. et al. 1983 “Modulation of Bordetella-Pertussis by Nicotinic-Acid.” Infection and Immunity 41(2): 516-522). The lack of nicotinic acid causes “Pellagra” which affects epithelia and nervous system. Moreover, nicotinamide and isonicotinamide as such were found to have antifungal and antimicrobial activity. (Shimai, T., Islam, M. T. et al. 2002 “Nicotinamide and structurally related compounds show halting activity against zoospores of the phytopathogenic fungus Aphanomyces cochlioides.” Zeitschrift Fur Naturforschung C-a Journal of Biosciences 57(3-4): 323-331; Sereno, D. Alegre, M. et al. 2005, “In vitro antileishmanial activity of nicotinamide.” Antimicrobial Agents and Chemotherapy 49(2): 808-812)

The present invention provides new Ag(I) complexes with derivatives of nicotinic acid, nicotinamide and related pyridine ligands.

The Ag(I) complexes according to the invention are preferably complexes with neutral ligands, which are structurally and chemically different compared to the Ag(I) complexes described e.g. in JP 2000016906, and are believed to have important advantages in terms of solubility, biological activity, bioavailability, and pharmacokinetics.

The present invention further provides methods for the use of Ag(I) complexes with derivatives of nicotinic acid and related pyridine ligands in the treatment, prevention and prophylaxis of infections.

The invention further provides a solvent-free method for the synthesis of Ag(I) complexes comprising substituted pyridines, pyrazines and pyrimidines as ligands, especially Ag(I) complexes comprising derivatives of nicotinic acid, nicotinamide and related pyridine as ligands.

### DETAILED DESCRIPTION OF THE INVENTION

The present invention provides a pharmaceutical preparation comprising, as an active ingredient, a compound according to Formula (I) or a pharmaceutical acceptable salt thereof optionally together with a pharmaceutical acceptable carrier for the use in the treatment, prophylaxis and prevention of infections,

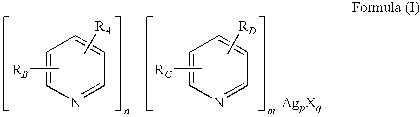

wherein n=1 to 4, m=1 to 4, p=1 to 4, and q=1 to 4,
R_{A} and R_{c} independently are H or NH₂,
R_{B} and R_{D} independently are

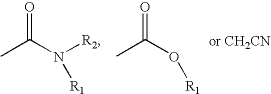

and, R₁ and R₂ independently are H, NO₂, alkyl, alkenyl, alkylcarbonyl, each optionally substituted with halogen,
with the proviso that if both R_{A} and R_{C} are H, then both R₁ and R₂ can not be H and where X is a negative counter-ion bonded or non-bonded to the Ag(I) ion.

X can be selected from NO₃⁻, HSO₄⁻, SO₄²⁻, HCO₃⁻, CO₃²⁻, H₃COO⁻, Cl⁻, Br⁻, I⁻, BF₄⁻, H₃PO₄⁻, H₂PO₄²⁻, and PO₄³⁻.

Preferably, the pharmaceutical preparation comprises a compound according to formula (I) wherein R₁ is NO₂, alkyl, alkenyl, alkylcarbonyl, each optionally substituted with halogen.

In other preferred embodiments the pharmaceutical preparation comprises a compound according to formula (I) wherein p=1, or even more preferably wherein n=1, m=1, p=1 and q=1.

In yet other preferred embodiments the pharmaceutical preparation comprises a compound according to formula (I) wherein R_{A} and R_{C} are identical and/or wherein R_{B} and R_{D} are identical.

In one preferred embodiment the present invention provides a pharmaceutical preparation comprising, as an active ingredient, a compound according to Formula (Ia) or a pharmaceutical acceptable salt thereof optionally together with a pharmaceutical acceptable carrier for the use in the treatment, prophylaxis and prevention of infections,

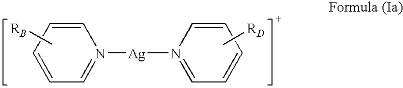

wherein R_{B} and R_{D} independently are

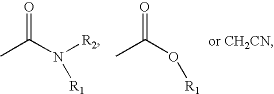

R₁ is NO₂, alkyl, alkenyl, alkylcarbonyl, each optionally substituted with halogen, and R₂ is H, NO₂, alkyl, alkenyl, alkylcarbonyl, each optionally substituted with halogen.

In one preferred embodiment the pharmaceutical preparation comprises a compound according to formula (Ia) wherein R_{B} and R_{D} are identical.

Preferably the compound is selected from
[Ag(ethylnicotinate)₂]X,
[Ag(ethylisonicotinato)₂]X,
[Ag(methylisonicotinate)₂],X, and
[Ag(2-pyridylacetonitrile)₂]X,
   where X is a negative counter-ion bonded or non-bonded to the Ag(I) ion.

X can be selected from NO₃⁻, HSO₄⁻, SO₄²⁻, HCO₃⁻, CO₃²⁻, H₃COO⁻, Cl⁻, Br⁻, I⁻, BF₄⁻, H₃PO₄⁻, H₂PO₄²⁻, and PO₄³⁻.

Most preferably the compound is selected from
Compound (2) [Ag(ethylnicotinate)₂]NO₃,
Compound (3) [Ag(ethylisonicotinato)₂]NO₃,
Compound (4) [Ag(methylisonicotinate)₂]NO₃, and
Compound (5) [Ag(2-pyridylacetonitrile)₂]NO₃.

In another preferred embodiment the present invention provides a pharmaceutical preparation comprising, as an active ingredient, a compound according to Formula (Ib) or a pharmaceutical acceptable salt thereof optionally together with a pharmaceutical acceptable carrier for the use in the treatment, prophylaxis and prevention of infections,

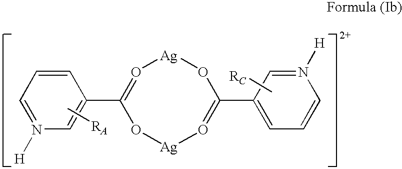

wherein R_{A} and R_{C} are NH₂ or H, and where at least one of R_{A} or R_{C} is NH₂.

Preferably the compound is selected from
[Ag₂-μ-O,O′(2-aminonicotinium)₂]X₂,
   where X is a negative counter-ion bonded or non-bonded to the Ag(I) ion.

X can be selected from NO₃⁻, HSO₄⁻, SO₄²⁻, HCO₃⁻, CO₃²⁻, H₃COO⁻, Cl⁻, Br⁻, I⁻, BF₄⁻, H₃PO₄⁻, H₂PO₄²⁻, and PO₄³⁻.

Most preferably the compound is
compound (6) [Ag₂-μ-O,O′(2-aminonicotinium)₂](NO₃)₂.

The present further provides a method for treatment, prophylaxis, or prevention of infections comprising administering to a mammal, including a human, in need thereof, a pharmaceutical effective amount of a compound according to Formula (I) or a pharmaceutical acceptable salt thereof,

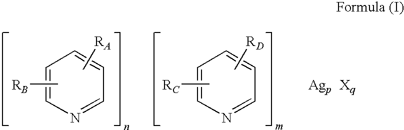

wherein n=1 to 4, m=1 to 4, p=1 to 4, and q=1 to 4,
R_{A} and R_{C} independently are H or NH₂,
R_{B} and R_{D} independently are

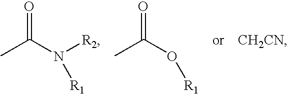

and, R₁ and R₂ independently are H, NO₂, alkyl, alkenyl, alkylcarbonyl, each optionally substituted with halogen,
with the proviso that if both R_{A} and R_{C} are H, then both R₁ and R₂ can not be H and where X is a negative counter-ion bonded or non-bonded to the Ag(I) ion.

X can be selected from NO₃⁻, HSO₄⁻, SO₄²⁻, HCO₃⁻, CO₃²⁻, H₃COO⁻, Cl⁻, Br⁻, I⁻, BF₄⁻, H₃PO₄⁻, H₂PO₄²⁻, and PO₄³⁻.

Preferably, the method comprises administering a compound according to formula (I) wherein R₁ is NO₂, alkyl, alkenyl, alkylcarbonyl, each optionally substituted with halogen.

In other preferred embodiments the method comprises administering a compound according to formula (I) wherein p=1, or even more preferably wherein n=l, m=1, p=1 and q=1.

In yet other preferred embodiments the method comprises administering a compound according to formula (I) wherein R_{A} and R_{C} are identical and/or wherein R_{B} and R_{D} are identical.

In one preferred embodiment the present invention provides a method for treatment, prophylaxis, or prevention of infections comprising administering to a mammal, including a human, in need thereof, a pharmaceutical effective amount of a compound according to Formula (Ia) or a pharmaceutical acceptable salt thereof,

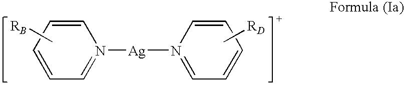

wherein R_{B} and R_{D} independently are

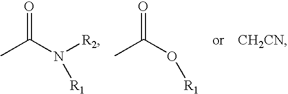

R₁ is NO₂, alkyl, alkenyl, alkylcarbonyl, each optionally substituted with halogen, and, R₂ is H, NO₂, alkyl, alkenyl, alkylcarbonyl, each optionally substituted with halogen.

In one preferred embodiment the method comprises administering a compound according to formula (Ia) wherein R_{B} and R_{D} are identical.

Preferably the compound is selected from
[Ag(ethylnicotinate)₂]X,
[Ag(ethylisonicotinato)₂]X,
[Ag(methylisonicotinate)₂],X, and
[Ag(2-pyridylacetonitrile)₂]X,
   where X is a negative counter-ion bonded or non-bonded to the Ag(1) ion.

X can be selected from NO₃⁻, HSO₄⁻, SO₄²⁻, HCO₃⁻, CO₃²⁻, H₃COO⁻, Cl⁻, Br⁻, I⁻, BF₄⁻, H₃PO₄⁻, H₂PO₄²⁻, and PO₄³⁻.

Most preferably the compound is selected from
Compound (2) [Ag(ethylnicotinate)₂]₃,
Compound (3) [Ag(ethylisonicotinato)₂]NO₃,
Compound (4) [Ag(methylisonicotinate)₂]NO₃, and
Compound (5) [Ag(2-pyridylacetonitrile)₂]NO₃.

In another preferred embodiment the present invention provides a method for treatment, prophylaxis, or prevention of infections comprising administering to a mammal, including a human, in need thereof, a pharmaceutical effective amount of a compound according to Formula (Ib) or a pharmaceutical acceptable salt thereof,

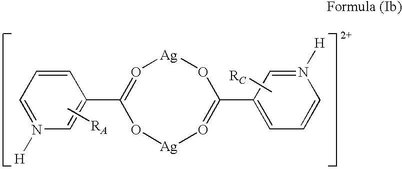

wherein R_{A} and R_{C} are NH₂ or H, and where at least one of R_{A} or R_{C} is NH₂.

Preferably the compound is selected from
[Ag₂-μ-O,O′(2-aminonicotinium)₂]X₂,
   where X is a negative counter-ion bonded or non-bonded to the Ag(I) ion.

X can be selected from NO₃⁻, HSO₄⁻, SO₄²⁻, HCO₃⁻, CO₃²⁻, H₃COO⁻, Cl⁻, Br⁻, I⁻, BF₄⁻, H₃PO₄⁻, H₂PO₄²⁻, and PO₄³⁻.

Most preferably the compound is
compound (6) [Ag₂-μ-O,O′(2-aminonicotinium)₂](NO₃)₂.

The present invention further provides use of a compound according to Formula (I) in the manufacture of a medicament for the use in the treatment, prophylaxis and prevention of infections,

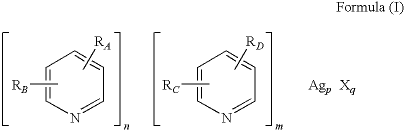

wherein n=1 to 4, m=1 to 4, p=1 to 4, and q=1 to 4,
R_{A} and R_{C} independently are H or NH₂,
R_{B} and R_{D} independently are

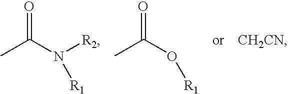

R₁ and R₂ independently are H, NO₂, alkyl, alkenyl, alkylcarbonyl, each optionally substituted with halogen,
with the proviso that if both R_{A} and R_{C} are H, then both R₁ and R₂ can not be H and where X is a negative counter-ion bonded or non-bonded to the Ag(I) ion.

X can be selected from NO₃⁻, HSO₄⁻, SO₄²⁻, HCO₃⁻, CO₃²⁻, H₃COO⁻, Cl⁻, Br⁻, I⁻, BF₄⁻, H₃PO₄⁻, H₂PO₄²⁻, and PO₄³⁻.

Preferably, the invention provides use of a compound according to formula (I) wherein R₁ is NO₂, alkyl, alkenyl, alkylcarbonyl, each optionally substituted with halogen.

In other preferred embodiments the invention provides use of a compound according to formula (I) wherein p=1, or even more preferably wherein n=l, m=1, p=1 and q=1.

In yet other preferred embodiments the invention provides use of a compound according to formula (I) wherein R_{A} and R_{C} are identical and/or wherein R_{B} and R_{D} are identical in the manufacture of a medicament for the use in the treatment, prophylaxis and prevention of infections.

In one preferred embodiment the present invention provides use of a compound according to Formula (Ia) in the manufacture of a medicament for the use in the treatment, prophylaxis and prevention of infections,

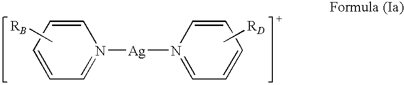

wherein R_{B} and R_{D} independently are

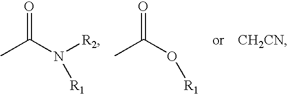

R₁ is NO₂, alkyl, alkenyl, alkylcarbonyl, each optionally substituted with halogen, and R₂ is H, NO₂, alkyl, alkenyl, alkylcarbonyl, each optionally substituted with halogen.

In one preferred embodiment the method comprises administering a compound according to formula (Ia) wherein R_{B} and R_{D} are identical.

Preferably the compound is selected from
[Ag(isonicotinamido)₂]X,
[Ag(ethylnicotinate)₂]X,
[Ag(ethylisonicotinato)₂]X,
[Ag(methylisonicotinate)₂],X, and
[Ag(2-pyridylacetonitrile)₂]X,
   where X is a negative counter-ion bonded or non-bonded to the Ag(I) ion.

X can be selected from NO₃⁻, HSO₄⁻, SO₄²⁻, HCO₃⁻, CO₃²⁻, H₃COO⁻, Cl⁻, Br⁻, I⁻, BF₄⁻, H₃PO₄⁻, H₂PO₄²⁻, and PO₄³⁻.

Most preferably the compound is selected from
Compound (1) [Ag(isonicotinamido)₂]NO₃,
Compound (2) [Ag(ethylnicotinate)₂]NO₃,
Compound (3) [Ag(ethylisonicotinato)₂]NO₃,
Compound (4) [Ag(methylisonicotinate)₂]NO₃, and
Compound (5) [Ag(2-pyridylacetonitrile)₂]NO₃.

In one preferred embodiment the present invention provides use of a compound according to Formula (Ib) in the manufacture of a medicament for the use in the treatment, prophylaxis and prevention of infections,

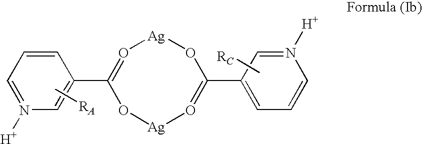

wherein R_{A} and R_{C} are NH₂ or H, and where at least one of R_{A} or R_{C} is NH₂.

Preferably the compound is selected from
[Ag₂-μ-O,O′(2-aminonicotinium)₂]X₂,
   where X is a negative counter-ion bonded or non-bonded to the Ag(I) ion.

X can be selected from NO₃⁻, HSO₄⁻, SO₄²⁻, HCO₃⁻, CO₃²⁻, H₃COO⁻, Cl⁻, Br⁻, I⁻, BF₄⁻, H₃PO₄⁻, H₂PO₄²⁻, and PO₄³⁻.

Most preferably the compound is
compound (6) [Ag₂-μ-O,O′(2-aminonicotinium)₂](NO₃)₂.

The present invention further provides novel compounds according to Formula (II),

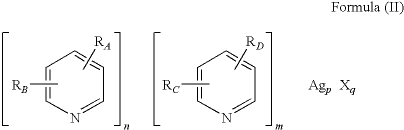

wherein n=1 to 4, m=1 to 4, p=1 to 4, and q=1 to 4,
R_{A} and R_{C} independently are H or NH₂,
R_{B} and R_{D} independently are

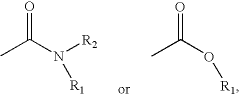

R₁ is NO₂, alkyl, alkenyl, alkylcarbonyl, each optionally substituted with halogen, and R₂ is H, NO₂, alkyl, alkenyl, alkylcarbonyl, each optionally substituted with halogen, and where X is a negative counter-ion bonded or non-bonded to the Ag(I) ion.

X can be selected from NO₃⁻, HSO₄⁻, SO₄²⁻, HCO₃⁻, CO₃²⁻, H₃COO⁻, Cl⁻, Br⁻, I⁻, BF₄⁻, H₃PO₄⁻, H₂PO₄²⁻, and PO₄³⁻.

In preferred embodiments the invention provides novel compounds according to formula (II) wherein p=1, or even more preferably wherein n=1, m=1, p=1 and q=1.

In yet other preferred embodiments the invention provides novel compounds according to formula (II) wherein R_{A} and R_{C} are identical and/or wherein R_{B} and R_{D} are identical.

In one preferred embodiment the present invention further provides novel compounds according to Formula (IIa)

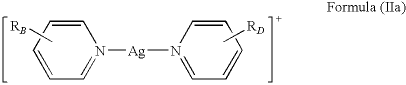

wherein R_{B} and R_{D} independently are

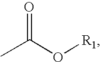

and R₁ is NO₂, alkyl, alkenyl, alkylcarbonyl, optionally substituted with halogen.

Preferably the compound is selected from
[Ag(ethylnicotinate)₂]X,
[Ag(ethylisonicotinato)₂]X,
[Ag(methylisonicotinate)₂],X, and
   where X is a negative counter-ion bonded or non-bonded to the Ag(I) ion.

X can be selected from NO₃⁻, HSO₄⁻, SO₄²⁻, HCO₃⁻, CO₃²⁻, H₃COO⁻, Cl⁻, Br⁻, I⁻, BF₄⁻, H₃PO₄⁻, H₂PO₄²⁻, and PO₄³⁻.

Most preferably the compound is selected from
Compound (2) [Ag(ethylnicotinate)₂]NO₃,
Compound (3) [Ag(ethylisonicotinato)₂]NO₃,
Compound (4) [Ag(methylisonicotinate)₂]NO₃, and

In another preferred embodiment the present invention further provides novel compounds according to Formula (IIb),

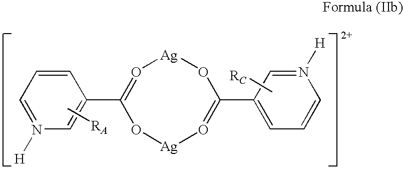

wherein R_{A} and R_{C} are NH₂ or H, and where at least one of R_{A} or R_{C} is NH₂.

Preferably the compound is selected from
[Ag₂-μ-O,O′(2-aminonicotinium)₂]X₂,
   where X is a negative counter-ion bonded or non-bonded to the Ag(I) ion.

X can be selected from NO₃⁻, HSO₄⁻, SO₄²⁻, HCO₃⁻, CO₃²⁻, H₃COO⁻, Cl⁻, Br⁻, I⁻, BF₄⁻, H₃PO₄⁻, H₂PO₄²⁻, and PO₄³⁻.

Most preferably the compound is
compound (6) [Ag₂-μ-O,O′(2-aminonicotinium)₂](NO₃)₂.

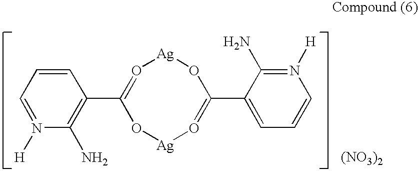

The present invention further provides a solvent-free method for the synthesis of Ag(I) containing compounds with substituted pyridines, pyrazines and pyrimidines as ligands, the method comprising the steps of

a) providing a Ag(I) salt, AgₙX
b) adding the ligand, and
c) mixing, grinding, kneading and/or melting the reaction mixture until full conversion to desired product;
where X can be selected from NO₃⁻, HSO₄⁻, SO₄²⁻, HCO₃⁻, CO₃²⁻, H₃COO⁻, Cl⁻, Br⁻, I⁻, BF₄⁻, H₃PO₄⁻, H₂PO₄²⁻, and PO₄³⁻. Preferably X is selected from NO₃⁻ and H₃COO⁻.

Preferably step b) comprises adding a stoichiometric amount of the ligand.

The method can further comprise the step of adding a catalytic amount of a suitable solvent.

In one preferred embodiment the present invention further provides a solvent-free method for the synthesis of compounds according to Formula (III),

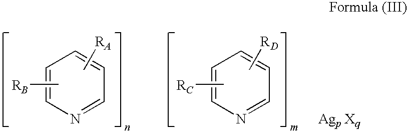

wherein n=1 to 4, m=0 to 4, p=1 to 4, and q=1 to 4,
R_{A} and R_{C} independently are H or NH₂,
R_{B} and R_{D} independently are H, CH₂CN or

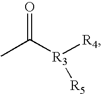

where R₃ is N or O,
and, if present, R₄ and R₅ independently are H, NO₂, alkyl, alkenyl, alkylcarbonyl, each optionally substituted with halogen,
and where X is a negative counter-ion bonded or non-bonded to the Ag(I) ion, the method comprising the steps of;
a) providing a Ag(I) salt, AgₙX
b) adding the ligand, and
c) mixing, grinding, kneading and/or melting the reaction mixture until full conversion to desired product;
where X can be selected from NO₃⁻, HSO₄⁻, SO₄²⁻, HCO₃⁻, CO₃²⁻, H₃COO⁻, Cl⁻, Br⁻, I⁻, BF₄⁻, H₃PO₄⁻, H₂PO₄²⁻, and PO₄³⁻. Preferably X is selected from NO₃⁻ and H₃COO⁻.

Preferably step b) comprises adding a stoichiometric amount of the ligand.

The method can further comprise the step of adding a catalytic amount of a suitable solvent.

### Pharmaceutical Use

As evidenced in Example 2, the compounds of the present invention show strong antimicrobial activity and arc useful in the treatment, prophylaxis and prevention of infections.

According to the present invention the infections to be treated or prevented are exemplified by, but not limited to, infections caused by bacteria, fungi, yeasts, or viruses, such as candidiasis, acne, herpes, and papilloma viral diseases.

The compounds of the present invention can be used in connection with treatment and prevention of pathological conditions of epithelial and dermal tissues, both intact and after lesion characterised by potential or acute infections sustained by pathogens, such as pathological conditions of the skin, of the mucosa and of the oral cavity, and external and internal genitals and ocular epithelia both intact and lesioned. In particular the compounds of the present invention can be used as therapeutic agents with disinfectant activity for the prevention, prophylaxis and treatment of the following pathological conditions:
Infections of the skin both intact and lesioned, of the oral mucosa, and external and internal genitals and ocular epithelia, brought about by bacteria, fungi, yeasts or viruses
Superficial or deep wounds, internal or external, grazes and abrasions, lacerated or contused wounds, wounds with soft tissue loss, strongly exudating wounds, both chronic and acute, surgical wounds, traumatic wounds
Superficial and deep burns
Vascular tropic lesions ischemic ulcers, vascular ulcers, diabetic ulcers, stasis ulcers, corneal ulcers
Bedsores
Athletes foot
Abscesses

In order to achieve a broad spectrum antimicrobial effect a mixture of two or more of the compounds of the present invention can be used.

In addition the compounds of the present invention can have useful applications in paraphysiological conditions and for preventive purposes in dermoprotective, lenitive and cosmetic parapharmaceutical preparations.

### Pharmaceutical Formulations

According to the pathology and the degree of seriousness the compounds of the present invention can be used in Ag(I) concentrations of 2·10⁻⁶ to 1·10⁻¹ mol/dm³ in topical formulations and in combination with appropriate diluents and helping substances compatible with the planned usage.

For all mentioned applications the compounds of the present invention can be present encapsulated in nanospheres or microspheres, be in the form of liquids, semi-solids, solids, containing excipients or diluents of pharmaceutical or cosmetic grade (for example solutions and aqueous, non-aqueous, hydroalcoholic suspensions, drops, gels, emulsions, creams, ovules, powder sprays, sprays with or without propellants, foams), be these new or known materials. Moreover, the compounds of the present invention can be supported, as they are or in any form mentioned above, upon inter biomaterials such as films, membranes, patches and dressings, also with slow release, or can be incorporated into biomaterials or into materials dissolving slowly or rapidly in aqueous environment.

### DEFINITIONS

As used throughout this specification and the appended claims, the following terms have the following meanings:

The term “alkyl” as used herein, means a straight or branched chain hydrocarbon containing from 1 to 6 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl.

The term “alkylcarbonyl” as used herein, means an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl.

The term “alkenyl” as used herein, means a straight or branched chain hydrocarbon containing from 2 to 6 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, and 5-hexenyl.

The term “carbonyl” as used herein, means a —C(═O)— group.

The term “halogen,” as used herein, means —Cl, —Br, —I or —F.

The present compounds can exist as pharmaceutical acceptable salts. The term “pharmaceutical acceptable salt,” refers to salts or zwitterions of the compounds which are water or oil-soluble or dispersible, suitable for treatment of disorders without undue toxicity, irritation, and allergic response, commensurate with a reasonable benefit/risk ratio, and effective for their intended use. The salts can be prepared during the final isolation and purification of the compounds or separately by reacting an amino group of the compounds with a suitable acid. Representative salts include acetate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetic, trifluoroacetic, glutamate, para-toluenesulfonate, undecanoate, hydrochloric, hydrobromic, sulfuric, phosphoric, and the like.

### EXAMPLES

### Example 1

### Synthesis of Ag(I) Compounds

The compounds (1) [Ag(isonicotinamido)₂]NO₃, (2) [Ag(ethylnicotinate)₂]NO₃, (3) [Ag(ethylisonicotinato)₂]NO₃, (4) [Ag(methylisonicotinate)₂]NO₃, (5) [Ag(2-pyridylacetonitrile)2]NO₃, (6) [Ag₂-μ-O,O′(2-aminonicotinium)₂](NO₃)₂, and (7) [Ag(isonicotinamido)₂]NO₃, were prepared from an aqueous silver nitrate solution and the corresponding free ligand dissolved in ethanol (route “a”), or by the direct, solvent free, combination of the silver salt and the ligand (route “b”).

The compounds were characterised by single crystal X-ray diffraction unambiguously proving structures in accordance with formulas (Ia) and (Ib), respectively.

### (1a) [Ag(isonicotinamido)₂]NO₃

To an aqueous solution (20 cm³) of AgNO₃ (0.34 g, 2.0 mmol) 0.49 g (4 mmol) of isonicotinamide in ethanolic solution (15 cm³) were added with continuous stirring. The clear mixtures were allowed to stand at room temperature for several days. Colourless crystals of [Ag(isonicotinamido)₂]NO₃ (1), suitable for X-ray measurement were collected, dried in air, with a yield ˜90% with respect to the metal. The compound is sparingly soluble in DMSO at about 0.003-0.005 g/ml. Analytical data for (1): Calc.: C, 34.97; H, 2.44; N, 16.99; Ag, 26.17%. Found: C, 34.84; H, 2.51; N, 17.09; Ag, 26.05%.

### (1b) [Ag(isonicotinamido)₂]NO₃, Solvent-Free Preparation

AgNO₃ (0.146 g, 0.86 mmol) and isonicotinamide (0.209 g, 1.7 mmol) were grinded in a mortar for 80 minutes. The resulting white powder had a powder X-ray diffraction pattern consistent with the calculated pattern from the single crystalline material 1 prepared according to 1a.

### (2a) [Ag(ethylnicotinate)₂]NO₂

To an aqueous solution (20 cm³) of AgNO₃ (0.34 g, 2.0 mmol) 0.60 g (4 mmol) of ethylnicotinate in ethanolic solution (15 cm³) were added with continuous stirring. The clear mixtures were allowed to stand at room temperature for several days. Colorless sheets of [Ag(ethylnicotinate)₂](NO₃) (2), suitable for X-ray measurement were collected, dried in air, with a yield ˜90% with respect to the metal. The compound is sparingly soluble in DMSO at about 0.003-0.005 g/ml. Analytical data for (2): Calc.: C, 40.69; H, 3.84; N, 8.90; Ag, 22.84%. Found: C, 40.73; H, 3.76; N, 8.97; Ag, 22.90%.

### (3a) [Ag(ethylisonicotinato)₂]NO₃

To an aqueous solution (20 cm³) of AgNO₃ (0.34 g, 2.0 mmol) 0.60 g (4 mmol) of ethylisonicotinate in ethanolic solution (15 cm³) were added with continuous stirring. The clear mixtures were allowed to stand at room temperature for several days. Colorless crystals of [Ag(ethylisonicotinato)₂(NO₃)] (3) suitable for X-ray measurement were collected, dried in air, with a yield ˜90% with respect to the metal. The compound is sparingly soluble in DMSO at about 0.003-0.005 g/ml. Analytical data for (3): Calc.: C, 40.69; H, 3.84; N, 8.90; Ag, 22.84%. Found: C, 40.55; H, 3.88; N, 9.01; Ag, 22.67%.

### (3b) [Ag(ethylisonicotinato)₂]NO₃ Solvent-Free Preparation

AgNO₃ (0.114 g, 0.67 mmol) and ethylisonicotinate (0.204 g, 1.35 mmol) were grinded in a mortar for 80 minutes. The resulting white powder had a powder X-ray diffraction pattern consistent with the calculated pattern from the single crystalline material 3 prepared according to 3a.

### (4a) [Ag(methylisonicotinate)₂]NO₃

To an aqueous solution (20 cm³) of AgNO₃ (0.34 g, 2.0 mmol) 0.54 g (4 mmol) of methylisonicotinate) in ethanolic solution (15 cm³) were added with continuous stirring. The clear mixtures were allowed to stand at room temperature for several days. Colorless crystals [Ag(methylisonicotinate)₂(H₂O)](NO₃) (4) suitable for X-ray measurement were collected, dried in air, with a yield ˜90% with respect to the metal. The compound is sparingly soluble in DMSO at about 0.003-0.005 g/ml. Analytical data for (4): Calc.: C, 36.38; H, 3.48; N, 9.09; Ag, 23.33%. Found: C, 36.45; H, 3.35; N, 9.20; Ag, 23.43%.

### (5a) [Ag(2-pyridylacetonitrile)₂]NO₃

To an aqueous solution (20 cm³) of AgNO₃ (0.34 g, 2.0 mmol) 0.47 g (4.0 mmol) of 2-pyridylacetonitrile in ethanolic solution (15 cm³) was added with continuous stirring. The clear mixtures were allowed to stand at room temperature for several days. Colorless crystals of [Ag(2-pyridylacetonitrile)₂]NO₃ (5) were collected, dried in air, with a yield 90% with respect to the metal.

### (6a) [Ag₂-μ-O,O′(2-aminonicotinium)₂](NO₃)₂,

To an aqueous solution (20 cm³) of AgNO₃ (0.34 g, 2.0 mmol) 0.27 g (2 mmol) of 2-aminonicotinic acid in ethanolic solution (15 cm³) were added with continuous stirring. White precipitate was formed, boiled, then filtered and the clear filtrate was allowed to stand at room temperature for several days. Colourless needles of the complex suitable for X-ray measurement were collected, dried in air, with a yield ˜90% with respect to the metal. Analytical data for (6): Calc. for C₁₂H₁₂Ag₂N₆O₁₀: C, 23.40; H, 1.96; N, 13.64; Ag, 35.02%. Found: C, 23.56; H, 1.71; N, 13.57; Ag, 35.02%.

### (7b) [Ag(isonicotinamido)₂]NO₃, Solvent-Free Preparation

AgNO₃ (0.17 g, 1 mmol) and 2-aminopyridine (0.28 g, 3 mmol) were grinded in a mortar for 80 minutes. The resulting white powder had a powder X-ray diffraction pattern consistent with the calculated pattern from the single crystalline material reported by Bowmaker (2005).

### Example 2

### Antimicrobial Activity

Their antimicrobial activity was established according to the recommendations of NCCLS, (National Committee for Clinical Laboratory Standards 1999, “Performance standards for antimicrobial susceptibility testing. NCCLS approved standard M100-S9” Wayne, Pa.) by the use of a broth microdilution method. Minimum inhibitory concentrations (MICs) for the tested compound were conducted using 12 different pathogens clinically isolated from diabetic foot ulcers and are all resistant strains for at least 10 antibiotics used for diabetic foot ulcer treatment. S. aureus 1, 2 and 3 and S. pyogenes 1, 2 and 3 as gram negative bacteria and P. mirabilis 1, 2 and 3 and Ps. aeruginosa 1, 2 and 3 as gram positive bacteria. The test materials were dissolved in DMSO. The highest concentration used was 256 μg/ml. The inoculum was 10⁵CFU/ml for bacteria and 10⁴CFU/ml for the yeast. Bacteria were cultured in Mueller Hinton Broth (MHB) for 24 h at 35° C. and the yeast in Glucose Peptone Broth (GPB) for 48 h at 30° C. MIC value was corresponding to the lowest concentration that inhibited the bacterial growth.

For the following seven samples, MICs values were determined and the antimicrobial activity is inversely proportional to this value. Concentrations used in this screening were: 1, 2, 4, 8, 16, 32, 64, 128 and 256 (μg/ml). 1 μg/ml corresponds to a 10⁻⁴% solution and an Ag(I) concentration of about 2·10⁻⁶ mol/dm³.

TABLE 1
Antimicrobial activity

| | Gram positive | | | | | |
|---|---|---|---|---|---|---|
| Compound | S. aureus 1 | S. aureus 2 | S. aureus 3 | S. pyogenes 1 | S. pyogenes 2 | S. pyogenes 3 |
| 1 | 16 | 32 | 32 | 64 | 128 | 256 |
| 2 | 16 | 4 | 4 | 2 | 4 | 2 |
| 3 | 8 | 4 | 16 | 32 | 16 | 16 |
| 4 | 16 | 32 | 8 | 64 | 128 | 64 |
| 6 | 32 | 32 | 64 | 128 | 256 | 256 |

| | Gram negative | | | | | |
|---|---|---|---|---|---|---|
| | | | | Ps. | Ps. | Ps. |
| Compound | P. mirabilis 1 | P. mirabilis 2 | P. mirabilis 3 | Aeruginosa 1 | Aeruginosa 2 | Aeruginosa 2 |
| 1 | 16 | 8 | 16 | 8 | 4 | 8 |
| 2 | 128 | 128 | 256 | 64 | 32 | 128 |
| 3 | 8 | 2 | 16 | 32 | 32 | 64 |
| 4 | 2 | 1 | 2 | 128 | 16 | 64 |
| 6 | 64 | 16 | 32 | 8 | 2 | 4 |

In a similar experiment compound (5) showed MIC values between 4 and 32, both for selected clinical isolates of gram positive and gram negative bacteria.

In a preliminary experiment compounds (1) to (6) were found to have antifungal activity, measured as the ability to inhibit growth of Candida albicans.

The above cited micro-organisms are provided as examples only and this list is not intended to define the scope and limits of the use of compounds of the present invention.

These results demonstrate that the tested compounds have strong antimicrobial activity, and furthermore demonstrate a specificity against certain strains, but also show that an appropriate mixture of the tested compounds could be used in blind chemotherapy against resistant bacteria.

1-30. (canceled)

31. A method for treatment, prophylaxis, or prevention of infections comprising administering to a mammal, including a human, in need thereof, a pharmaceutical effective amount of a compound according to Formula (I) or a pharmaceutical acceptable salt thereof, 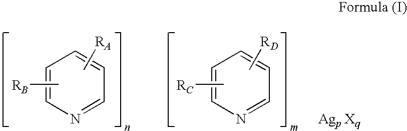
wherein n=1 to 4, m=1 to 4, p=1 to 4, and q=1 to 4,
R_{A} and R_{C} independently are H or NH₂,
R_{B} and R_{D} independently are 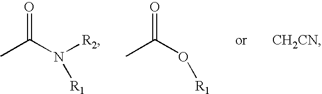
and R₁ and R₂ independently are H, NO₂, alkyl, alkenyl, alkylcarbonyl, each optionally substituted with halogen,
with the proviso that if both R_{A} and R_{C} are H, then both R₁ and R₂ can not be H and where X is a negative counter-ion bonded or non-bonded to the Ag(I) ion.

32. A method according to claim 31 where the a compound is a compound according to Formula (I) wherein R₁ is NO₂, alkyl, alkenyl, alkylcarbonyl, each optionally substituted with halogen.

33. A method according to claim 31 where the compound is a compound according to Formula (Ia), 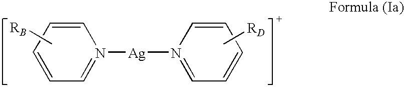
wherein R_{B} and R_{D} independently are 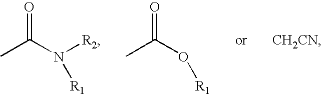
R₁ is NO₂, alkyl, alkenyl, alkylcarbonyl, each optionally substituted with halogen and, R₂ is are H, NO₂, alkyl, alkenyl, alkylcarbonyl, each optionally substituted with halogen.

34. A method according to claim 33 where the compound is a compound selected from
[Ag(ethylnicotinate)₂]NO₃,
[Ag(ethylisonicotinato)₂]NO₃,
[Ag(methylisonicotinate)₂]NO₃, and
[Ag(2-pyridylacetonitrile)₂]NO₃.

35. A method according to claim 31 where the compound is a compound according to Formula (Ib), 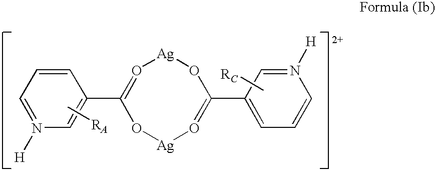
wherein R_{A} and R_{C} are NH₂ or H, and where at least one of R_{A} or R_{C} is NH₂.

36. A method according to claim 35 where the compound is the compound
[Ag₂-μ-O,O′(2-aminonicotinium)₂](NO₂)₂.

37. A compound according to Formula (II), 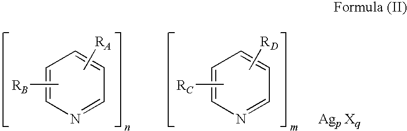
wherein n=1 to 4, m=1 to 4, p=1 to 4, and q=1 to 4,
R_{A} and R_{C} independently are H or NH₂,
R_{B} and R_{D} independently are 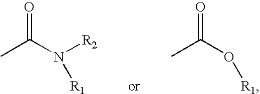
R₁ is NO₂, alkyl, alkenyl, alkylcarbonyl, each optionally substituted with halogen and, R₂ is H, NO₂, alkyl, alkenyl, alkylcarbonyl, each optionally substituted with halogen, and where X is a negative counter-ion bonded or non-bonded to the Ag(I) ion.

38. A compound according to Formula (IIa) 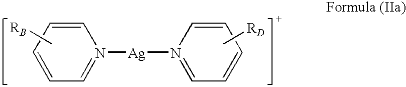
wherein R_{B} and R_{D} independently are 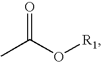
and R₁ is NO₂, alkyl, alkenyl, alkylcarbonyl, optionally substituted with halogen.

39. A compound according to claim 38 selected from
Compound (2) [Ag(ethylnicotinate)₂]NO₃,
Compound (3) [Ag(ethylisonicotinato)₂]NO₃,
Compound (4) [Ag(methylisonicotinate)₂]NO₃, and

40. A compound according to Formula (IIb), 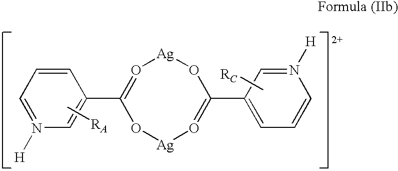
wherein R_{A} and R_{C} are NH₂ or H, and where at least one of R_{A} or R_{C} is NH₂.

41. A compound according to claim 40 which is the compound
[Ag₂-μ-O,O′(2-aminonicotinium)₂](NO₃)₂.

42. A solvent-free method for the synthesis of Ag(I) containing compounds with substituted pyridines, pyrazines and pyrimidines as ligands, the method comprising the steps of;
a) providing a Ag(I) salt, AgₙX
b) adding the ligand, and
c) mixing, grinding, kneading and/or melting the reaction mixture until full conversion to desired product;
where X can be selected from NO₃⁻, HSO₄⁻, SO₄²⁻, HCO₃⁻, CO₃²⁻, H₃COO⁻, BF₄⁻, H₃PO₄⁻, H₂PO₄²⁻, PO₄³⁻.

43. The method according to claim 42 where step b) comprises adding a stoichiometric amount of the ligand.

44. The method according to claim 42 where X is selected from NO₃⁻ and H₃COO⁻.

45. The method according to claim 42 where the Ag(I) containing compound is a compound according to Formula (III), 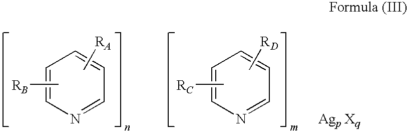
wherein n=1 to 4, m=0 to 4, p=1 to 4, and q=1 to 4,
R_{A} and R_{C} independently are H or NH₂,
R_{B} and R_{D} independently are H, CH₂CN or 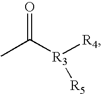
where R₃ is N or O,
and, if present, R₄ and R₅ independently are H, NO₂, alkyl, alkenyl, alkylcarbonyl, each optionally substituted with halogen,
and where X is a negative counter-ion bonded or non-bonded to the Ag(I) ion;
the method comprising the steps of;
a) providing a Ag(I) salt, AgₙX
b) adding the ligand, and
c) mixing, grinding, kneading and/or melting the reaction mixture until full conversion to desired product;
where X is selected from NO₃⁻, HSO₄⁻, SO₄²⁻, HCO₃⁻, CO₃²⁻, H₃COO⁻, Cl⁻, Br⁻, I⁻, BF₄⁻, H₃PO₄⁻, H₂PO₄²⁻, and PO₄³⁻.

46. The method according to claim 45 where step b) comprises adding a stoichiometric amount of the ligand.

47. The method according to claim 45 where X is selected from NO₃⁻ and H₃COO⁻.

48. The method according to claim 42 further comprising the step of adding a catalytic amount of a suitable solvent.
